(19) United States
(12) Patent Application Publication
(10) Pub. No.: US 2006/0014155 A1
(43) Pub. Date: Jan. 19, 2006

(54) Methods for the production of sensor arrays using electrically addressable electrodes

(75) Inventors: Robert J. Hamers, Madison, WI (US); Sarah Baker, Madison, WI (US); Chang-Soo Lee, Daejeon (KR)

Correspondence Address: FOLEY & LARDNER LLP, 150 EAST GILMAN STREET, P.O. BOX 1497, MADISON, WI, 53701-1497, US

(73) Assignee: Wisconsin Alumni Research Foundation

(21) Appl. No.: 10/892,819

(22) Filed: Jul. 16, 2004

(51) Int. Cl. C12Q 1/68

(52) U.S. Cl. 435/6; 205/777.5

(57) ABSTRACT

Methods for building sensor arrays using electrical signals to selectively functionalize individual electrodes in an array of electrically addressable electrodes are provided. These methods are useful for providing sensor arrays for use in chemical and biochemical assays. The method is based on the sequential electrochemical reduction of functional groups on individual electrodes in order to selectively promote the functionalization of selected electrodes with selected binding entities.

### STATEMENT OF GOVERNMENT RIGHTS

Research funding was provided for this invention by the National Science Foundation under grant Nos. NSF: 0210806; 0314618; 0330257; and 0079983. The United States government has certain rights in this invention.

### FIELD OF THE INVENTION

The present invention relates generally to the field of microarrays for chemical and biochemical assays and methods for their production.

### BACKGROUND

Sensors designed to determine the presence of molecules, including biomolecules, are commonly used in the chemical and biotechnology industries today to perform rapid chemical and biochemical analysis. These sensors are adapted to detect and/or quantify various analytes based on known interactions between the analytes and binding entities immobilized on a substrate. In some instances, the sensors may be provided in the form of microsensors where all of the binding entities are bound to a microscopically small area on a chip. Many of the sensors today are designed to detect and/or quantify multiple analytes in one assay by using a variety of different binding entities immobilized on a single substrate. In these types of assays, each binding entity desirably occupies a selected spatial region of the sensor, thereby allowing each binding entity to be distinguished from the others. Unfortunately, using many of the fabrication processes available today, the time required to selectively spatially bind the various binding agents to the substrate can be quite long.

Several methods have been proposed for fabricating sensor arrays. For example, some conventional methods involve placing individual spots of binding entities, such as biomolecules, on precise locations on a sensor chip using either a microspotter or by using microfluidic handling to flow the binding entities to a precise location on the chip. Unfortunately, this process can be complicated, costly and very time consuming. More recently the spotting techniques have been advanced to employ inkjet printing technology to dispense spots of binding entities onto specific sites of the substrate. However, this inkjet spotting technique tends to be slow and is well-suited only for lower volume sensor array fabrication.

Other methods for the fabrication of sensor arrays involve the use of electrochemical patterning to provide for the step-wise chemical synthesis of binding entities on a substrate. In these methods, an array of individually electrically addressable electrodes is provided on a substrate and the potential of one or more electrodes in the array is altered in order to deposit, remove or chemically modify molecules near or in contact with the electrode. For example, U.S. Pat. No. 6,203,758 discloses a microcircuit for performing biomolecular analysis which includes at least one microelectrode having a protective layer applied thereon. When the microelectrode is electrically activated, the protective layer is removed, allowing a binding entity to bond to the underlying microelectrode. U.S. Pat. No. 6,251,595 discloses a sensor composed of a plurality of electrodes supported by a semiconductor substrate wherein the electrodes are initially derivatized with protecting groups that render the electrodes chemically inert. These protecting groups may be removed by selectively activating the electrodes to which they are bound. Once the protecting groups have been removed the electrodes are free to react with various binding entities. Similarly, U.S. patent application Publication No. 2003/0224387 discloses a biosensor composed of an array of electrodes each of which is initially overlaid by protective molecules which inhibit binding entities from binding to the electrodes. Each electrode may be selectively deprotected by applying a potential to that electrode to dissociate the protecting groups. The deprotected electrodes may then be exposed to binding entities with which they react to form a sensor array.

None of the above-mentioned references discloses a device in which functional groups on individually electrically addressable electrodes undergo electrochemical reduction to provide a new set of functional groups that react with and immobilize binding entities.

### SUMMARY OF THE INVENTION

Sensor arrays fabricated using an array of electrically addressable electrodes and methods for producing the arrays are provided. The methods are designed to selectively modify individual electrodes in an array of electrically addressable electrodes using the selective and sequential electrochemical reduction of functional groups on individual electrodes or sets of electrodes in the array. This selective electrochemical reduction of derivatized electrodes activates the selected electrodes, rendering them reactive toward binding entities.

The methods provided herein generally include the steps of selectively modifying electrodes derivatized with a first functional group by applying a negative reducing potential to at least one electrically addressable electrode in an array of electrodes in order to electrochemically reduce the first functional group (e.g. a nitro group) to provide a second functional group (e.g. an amino group). This reduction step is followed by selectively immobilizing a binding entity on the selected electrode or electrodes by exposing the second functional groups to a binding entity that is reactive with the second functional group but not with the first functional group.

The cycle of selective reduction followed by binding entity immobilization may be repeated, typically using different binding entities in each cycle, until each of the electrodes in the array has a selected binding entity immobilized thereon.

The electrically addressable electrodes are desirably made from a carbon-containing material. In one embodiment the electrodes are made from electrically conductive contacts on which carbon nanotubes have been attached or grown. Other suitable carbon-containing materials include, diamond, glassy carbon, electrically conducting polymers and diamond like carbon.

The binding entities are made from sensor molecules that exhibit specific affinities for analyte molecules that may be of interest to the user. The sensor molecules may be various synthetic molecules or biomolecules. The sensor molecules may themselves be functionalized with a functional group that is capable of reacting with the second functional groups on the electrodes in the array. Alternatively, the sensor molecules may be bound to spacer molecules that are capable of reacting with the second functional groups. In the latter embodiment it is the reaction product of the sensor molecule and the spacer molecule that make up the binding entity.

Further objects, features and advantages of the invention will be apparent from the following detailed description when taken in conjunction with the accompanying drawings.

### BRIEF DESCRIPTION OF THE DRAWINGS

FIG. 1 is schematic diagram of a stepwise procedure for making a sensor array.

FIG. 2 shows a reaction scheme that may be used to provide a biosensor array for detecting oligonucleotides.

FIG. 3 is a greyscale rendition of a fluorescence image obtained from a selectively functionalized sensor array as described in Example 1.

FIG. 4a is a scanning electron microscopy image of a vertically aligned carbon nanotube bundle that may be used to provide an individually electrically addressable electrode.

FIG. 4b is a white-light image of six Mo electrodes having vertically aligned carbon nanotube bundles grown thereon.

FIG. 4c is a fluorescence image of the six Mo electrodes of FIG. 4b showing only those electrodes that were selectively electrochemically treated and functionalized.

### DETAILED DESCRIPTION OF THE INVENTION

A method of building a sensor array using electrical signals to selectively functionalize individual electrodes in an array of electrically addressable electrodes is provided. These methods are useful for providing sensor arrays for use in chemical and biochemical assays. The method is based on the sequential electrochemical reduction of functional groups on individual electrodes in order to selectively promote the functionalization of selected electrodes with selected binding entities. These methods provide a way to functionalize very small array elements in a high density array without the need to use microfluidics or other difficult fluid handling operations.

The methods provided herein are designed to selectively modify individual electrodes in an array of electrically addressable electrodes. Each of the electrodes in the array is initially derivatized with a first functional group which is nonreactive with respect to the binding entities that have been chosen to form the sensor array. By selectively and sequentially applying a negative potential to each of the electrically addressable electrodes in the array (or, alternatively, to individual sets of electrically addressable electrodes in the array), the first functional groups may be selectively and sequentially electrochemically reduced to provide second functional groups which are reactive with chosen binding entities. Thus, by selectively reducing the first functional groups on one electrode (or one set of electrodes), in the array and then exposing the array to a given binding entity, the user may selectively react said binding entity with only that electrode (or that set of electrodes) to which the reducing potential was applied. The steps of selectively applying a potential to the electrodes followed by exposing the electrodes to a chosen binding entity may be repeated until each of the electrodes in the array has been selectively functionalized with a binding entity.

The devices used to carry out the present methods generally include a substrate having an array of electrically addressable electrodes disposed thereon. The substrate may be any convenient substrate capable of electrically isolating the electrodes. In some instances the substrate is desirably a semiconductor substrate, such as a silicon wafer, so that the production of the devices may take advantage of well developed semiconductor processing equipment and techniques. However, other substrates may be used. Examples of suitable substrates include but are not limited to glass substrates, ceramic substrates, silicon dioxide substrates and plastic substrates.

The array of electrodes disposed on the substrate will include at least two electrically addressable electrodes. The electrodes themselves may take on a variety of shapes and sizes and may be arranged in an array having a regular or irregular pattern. However, in many instances it is desirable to use electrodes with small dimensions which are separated by small gaps so that a densely packed array may be patterned onto a very small surface area to produce a microarray. For example, in some embodiments the electrodes and the spacing between electrodes will have dimensions of no more than about 100 microns (e.g., between about 5 and 100 microns). In other embodiments the dimensions of the electrodes and the spacing between electrodes may be much smaller. For example, in some instances these dimensions may be no more than about 5 microns, no more than about 2 microns, no more than about 1 micron or no more than about 0.5 micron. Such small dimensions may be produced using optical lithography, x-ray lithography or electron beam lithography techniques. The electrode arrays may include any number of electrodes (e.g. at least 10, at least 100 or at least 1000.) However, the use of small electrodes makes it possible to fit tens of thousands (i.e. at least 10,000) or even hundreds of thousands (i.e. at least 100,000) of microelectrodes on a single small substrate chip. The present methods are particularly well-suited for use in the production of arrays having such small length scales because the electron-transfer step in the electrochemistry can typically only occur for species within about 1 nm of an electrode surface. Thus, in some instances the present methods may be used to produce selectively functionalized arrays having sub-micron, near-atomic length scales.

Carbon containing electrodes are examples of electrodes that may be employed in the devices disclosed herein. Carbon containing electrodes are favored because they may be readily derivatized with a variety of useful functional groups. Carbon containing electrodes include carbon nanotube electrodes, carbon fiber electrodes, electrodes made from elemental carbon and electrically conductive polymer electrodes. Electrically conductive polymers that may be used in the present devices include, but are not limited to, pentacene, polypyrrole, polyaniline and polythiophene. Electrodes made from elemental carbon include, diamond electrodes, glassy carbon electrodes, diamond-like carbon electrodes and graphitic carbon electrodes. Examples of suitable graphitic carbon electrodes include the thin-film carbon electrodes made from the pyrolysis of photoresist films described in Blackstock et al., Anal. Chem., 76, (3), 544-2552 (2004) and Ranganathan et al., Anal. Chem., 73, (5), 893-900 (2001), the entire disclosures of which are incorporated herein by reference.

Generally, the electrodes may be deposited in an array directly onto the substrate. However, in some instances, such as when carbon nanotubes are used as electrode materials, an array of electrically conductive contacts may be deposited in an array on the substrate and the electrode material may be disposed over the contacts.

Carbon nanotubes are well-suited for use as electrode materials because studies have shown that carbon nanotubes may be covalently modified with biomolecules such as DNA and that the biomolecules immobilized on the carbon nanotubes retain their biomolecular recognition capabilities. When carbon nanotubes are used to provide the array of electrodes, an array of conductive contacts is first deposited onto a substrate and the carbon nanotubes are subsequently deposited or grown on the contacts. Suitable conductive contacts include metal contacts such as molybdenum contacts. One illustrative method for growing single walled carbon nanotubes on molybdenum contacts is described in Example 1 below. As used herein the term “carbon nanotube” is used broadly to refer to carbon nanotubes, as well as other generally cylindrically-shaped carbon nanostructures, such as nanowires, nanowhiskers, nanofibers (e.g. vertically aligned carbon nanofibers), nanofilaments and the like.

The dimensions of small carbon fibers, and specifically vertically aligned carbon nanofibers, make them particularly well-suited for use in constructing electrodes in the present electrode arrays. This is because a bundle of vertically aligned carbon fibers disposed on a small planer electrode provides a relatively high density of binding sites for binding entities along the length of the fibers, yet because they are vertically aligned on the electrode, the bundles take up a very small area on the electrode surface. As a result, very small, densely packed sensor arrays may be produced. In some such sensor arrays, the functionalized regions on the electrodes (e.g. the surface area occupied by a binding-entity-funtionalized carbon nanofibers) may be no more than about 1 microns. This includes embodiments where the functionalized region on an electrode has a surface area of no more than about 0.5 microns and further includes embodiments where the functionlized region on an electrode has a surface area of no more than about 0.25 micron.

The ability to provide sub-micron biologically-modified regions (e.g. binding entity-funtionalized regions) on a substrate surface is highly desirable. For example, in some experiments a user may want to conduct measurements on a single cell. For these experiments, a surface functionalized with different receptors that are closer together than the size of a single cell is desirable. The present methods make the fabrication of such surfaces possible. In some embodiments, the present methods provide a modified surface composed of a substrate surface (e.g. an electrode surface) having at least two surface regions each functionalized with a binding-entity, wherein the functionalized surface regions have surface areas (i.e. the area of the substrate surface which they occupy) of less than 1 micron and further wherein the binding entities of the at least two regions are separated by less than about 1 micron. This includes embodiments where the binding entity-functionalized surface regions have a surface area of no more than 0.5 microns and/or the binding entities of the at least two regions are separated by less than about 0.5 microns.

Deposition of the array of electrodes or contacts onto a substrate may be carried out by patterning the substrate using standard lithographic (e.g., photolithographic) techniques and simple mask designs. For example, a silicon wafer substrate coated with a layer of silicon dioxide may be coated with a photoresist, masked with a circuitry pattern, exposed and developed. A layer of electrode or contact material may then be deposited to form the circuitry. The remaining resist pattern is then removed, leaving a circuitry pattern including the electrodes and/or contacts and their connective wires patterned on the surface of the wafer. Another, more detailed, exemplary procedure for fabricating a substrate having an array of microelectrodes deposited thereon is described in Example 1 below.

In some embodiments of the devices, every electrode in the array will be individually addressable. In other embodiments of the devices, the electrodes in the array will be divided into a plurality of sets of electrodes wherein each set may be collectively addressable.

Once the array of electrically addressable electrodes is deposited onto a substrate, the electrodes may be derivatized with a first functional group that is nonreactive with one or more binding entities selected for immobilization on the substrate. Nitro (NO₂) groups (including both nitroaromatic and nitroalkyl groups) are well suited for use as the first functional group. The chemistry used to derivatize the electrodes with the first functional group will vary depending on the material from which the electrodes are constructed. In some cases, the derivatization of carbon containing substrates may be carried out using known chemical reaction schemes. When the electrodes are made from carbon nanotubes, carbon fibers or diamond thin films, diazonium chemistry may be used to link aromatic nitro groups to the nanotubes. A reaction scheme for carrying out such diazonium chemistry on carbon nanotubes is discussed in greater detail in Example 1 below. A reaction scheme describing the derivatization of a diamond electrode with nitro groups is described in Example 2 below. An electrochemical scheme for derivatizing carbon nanotubes with nitrophenyl groups is described in Kooi et al., Angew. Chem. Int. Ed., 41, 1353-1355 (2002), the entire disclosure of which is incorporated herein by reference.

The electrochemical reduction of the first functional groups may be carried out by applying a negative reducing potential to one of the electrodes (or a set of electrodes) in the array. The electrodes, other than those actually activated at a given time, can be used as the counter electrodes. Additionally, or alternatively, an external counter electrode may be employed. The potential should be large enough to induce the electrochemical reduction and may vary depending upon the nature of the electrode and the first functional group. In some embodiments a potential of about −0.1 to −2 volts is sufficient. For example, the reduction of nitro groups on an electrode comprising carbon nanotubes disposed on a molybdenum contact may generally be carried out by applying a potential of about −1 to −1.5 V.

Once the first functional groups on one or more of the electrodes in the array of electrically addressable electrodes have been reduced to provide one or more electrodes derivatized with a second functional group that is reactive with a selective binding entity, those electrodes may be exposed to the selected binding entity under conditions which promote a reaction between the binding entity and the second functional groups. For example, the device may be immersed in a solution containing the selected binding entity under conditions that promote reaction and covalent bond formation.

In the case where the second functional groups comprise amino groups, produced via the electrochemical reduction of nitro groups, the devices may be exposed to binding entities having a group that is reactive with the amino groups. Examples of such reactive groups include aldehyde groups, epoxide groups, chloracid groups, carboxylic acid groups and ester groups. Reactions between primary amino groups and the above-listed groups employed in the fabrication of biosensor arrays are known. For example, descriptions of reaction schemes for immobilizing biomolecules, such as DNA molecules, antibodies and nanostructures, on amino terminated substrates, including diamond and glassy carbon substrates may be found in Yang et al., Nature Materials, 1, 253-257 (2002); Strother et al., J.A.C.S., 122, 1205-1209 (2000); and Baker et al., Science, 293, 1289-1292 (2001), the entire disclosures of which are incorporated herein by reference. A specific example of a reaction scheme that may be used to immobilize DNA molecules on a amino functionalized substrate is provided in Example 1 below.

The binding entities which are immobilized on the substrates through reactions with the second functional groups will include a sensor molecule (e.g., a biomolecule or a synthetic molecule) having a specific affinity for an analyte molecule. Thus, when the sensor arrays provided herein are exposed to the appropriate analyte molecules, those analyte molecules will bind to the sensor molecules of the binding entities through covalent or noncovalent interactions. Sensor molecules that may be used as binding entities or as parts of binding entities include, but are not limited to, DNA molecules, RNA molecules, synthetic oligonucleotides, peptides, polypeptides, proteins, enzymes, antibodies, receptors, polysaccharides, synthetic polymers, ligands and viruses. Thus, by way of example, binding entities comprising oligonucleotides may bind to analytes comprising complimentary oligonucleotides through oligonucleotide hybridization interactions. Similarly, binding entities including antibody sensor molecules may be used to detect antigen analytes via antibody-antigen interactions.

In some instances the sensor molecules will themselves include functional groups capable of reacting with and bonding to the second functional groups on the electrode arrays. More commonly, however, the binding entities will be composed of sensor molecules that have been functionalized with an appropriate functional group to provide reactivity and bonding between the binding entity and the second functional groups or the electrode array. In some such instances, the binding entities will include a spacer molecule attached to the sensor molecule. In these embodiments it is the spacer molecule that includes a reactive functionality capable of reacting with the second functional groups on the electrodes of the array. The spacer molecules may serve to properly orient the sensor molecule of the binding entity for interaction with the analyte molecules. Additionally, in cases where the sensor molecules are bioactive biomolecules, such as enzymes, the spacer molecules may be used to optimize the spacing between the substrates and the sensor molecules so that the biomolecules retain their bioactivities. In some instances sensor molecules which are immobilized on a substrate via an appropriate spacer molecule may have a bioactivity approaching that of the free biomolecule.

In the fabrication of the sensor arrays, the spacer molecules may first be reacted with the second functional groups on the electrodes of the array, followed by the reaction of the sensor molecules with the immobilized spacer molecules, such that the binding entities are built up in a stepwise fashion on the electrode array. Alternatively, the spacer molecules may first be reacted with the sensor molecules and the resulting intact binding entities may subsequently be immobilized on the electrode array.

The step-wise process of selective reduction of one or more electrodes in the electrode array followed by exposure of the selected electrodes to a binding entity in order to functionalize the selected electrodes may be repeated using a different binding entity in each cycle to produce arrays having a number of different binding entities immobilized thereon. For example, in some instances each electrode in an array of electrically addressable electrodes will be functionalized with a different type of binding entity.

The following illustrative embodiments are intended to further exemplify the methods for producing biosensor arrays. These embodiments should not be interpreted as limiting the scope of the methods provided herein.

### EXAMPLES

### Example 1

### Production of a Microsensor Array from an Array of Carbon Nanotube Electrodes

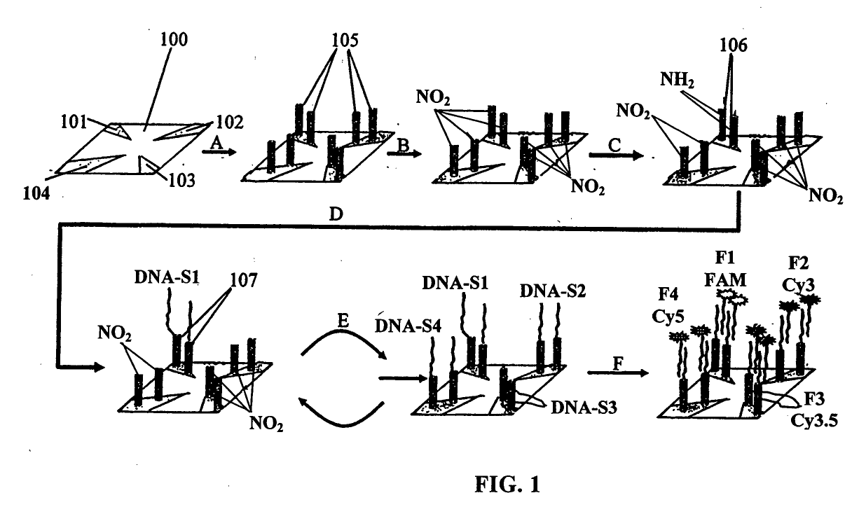

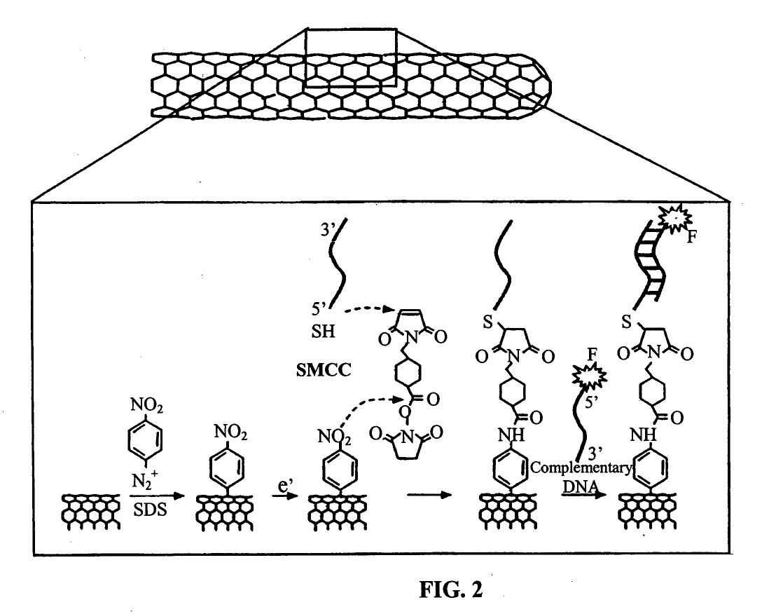

This example describes the production of a biosensor array using an array of individually electrically addressable electrodes made from carbon nanotubes grown on molybdenum (Mo) contacts. The stepwise process outlined in detail below is shown schematically in FIG. 1. A more detailed reaction scheme, showing the reaction chemistry is shown in FIG. 2.

Substrate Patterning:

A Silicon <100> wafer 100 with a resistivity of 0.01 to 0.02 ohm-cm and a 0.5 [symbol] m thermal oxide grown on both sides was used as the substrate. The nanotube contact pads were patterned in Shipley 1813 photoresist using G-line photolithography on a Canon PLA-501 contact aligner. The pattern was developed using Shipley MF 321. Fifty nanometers of molybdenum were evaporated onto the substrate to form the desired pattern using a Telemark CHA-600 e-beam evaporator. Liftoff was done in acetone. The nanotube catalyst was patterned onto the molybdenum electrodes using electron beam lithography according to the following procedure. First, a formulation of polymethyl methacrylate (PMMA) (2%, 950 K molecular weight, in anisole) resist (Microchem) was spin coated onto the substrate at 4,000 RPM. This positive radiation resist was then exposed at select regions using a LEO Supra 55 VP SEM equipped with a Nabity electron beam lithography system and a dose of 220 [symbol] C/cm² at 25 kV. The exposed sites over the microelectrodes were then selectively removed using a developer consisting of a 1:3 solution of methyl isobutyl ketone and isopropyl alcohol (Microchem), leaving a set of patterned Mo electrodes 101-104.

Nanotube Growth:

Single-walled carbon nanotubes 105 were grown on the Mo electrodes via catalyzed thermal chemical vapor deposition (shown as step A in FIG. 1). The catalyst, an alumina-supported Mo/Fe nanoparticle catalyst (First Nano) was spin-coated onto the substrate at 3,000 RPM; liftoff of the excess catalyst and resist was done in N-methyl pyrrolidone (Acros chemicals.) at a temperature of 60° C. Finally, the patterned chip was placed into a commercially available nanotube growth furnace (First Nano). The chemical vapor deposition was carried out using 400 sccm methane as the carbon feedstock and 20 sccm hydrogen as the diluent gas so that the Mo contact pads would not oxidize. The growth temperature was 900° C. and growth time was 10 minutes. Raman spectroscopy confirmed that single-walled carbon nanotubes with diameters of approximately 1 nm covered the Mo electrodes.

Nanotube Functionalization with Aromatic Nitro Groups Via Diazonium Chemistry (Shown as Step B in FIG. 1):

In order to functionalize the electrically-addressable carbon nanotubes 105 on the chip 100 with nitro groups, the chip was immersed in a 36 mM solution of 4-nitrobenzenediazonium tetrafluoroborate (Aldrich) in 1% sodium dodecyl sulfate (Promega) and shaken using a vortex mixer at room temperature for 24 hours. The chip was then rinsed by immersing and gently shaking in three fresh solutions each of deionized (DI) water and acetone. At this stage, the chip consisted of Mo electrodes coated with single-walled carbon nanotubes bearing reactive nitrophenyl groups.

Electrically-Addressable Electrochemical Modification of Specific Nanotube-Coated Mo Electrodes:

In order to electrochemically “activate” a specific set of carbon nanotubes on the chip by converting the nitro groups to amino groups, the molybdenum contact associated with these nanotubes was connected to a potentiostat (Solartron) as the working electrode; the other nanotube-covered Mo contacts and a larger Pt foil were used as the counter-electrode, and a AgCl-coated Ag wire as the reference electrode. The reduction of the nitro-benzene groups on the carbon nanotubes was simultaneously monitored and carried out using cyclic voltammetry on a Solartron Si 1287 electrochemical interface using the Corrware software package. The potential (reported vs. the Ag/AgCl reference electrode) was swept from a starting potential of −1.0 V down to −1.8 V, up to −0.2 V, and back to −1.0 V, at a rate of 200 mV/sec in a solution of 0.1 M KCl in ethanol:DI water 90:10. As the potential was brought negative, the nitro groups were reduced to amino groups at a potential of about −1.0 to −1.5, leading to a small reduction peak in the cyclic voltammogram. As the potential was brought more positive, no corresponding oxidation peak was observed, demonstrating that the amino groups are not easily reoxidized. Typically, each scan was repeated four times in order to verify the irreversible conversion of nitro-terminated nanotubes to amino-terminated nanotubes. This step produces reactive amino groups only on the specific subset of nanotubes 106 that are on the Mo electrode that was used as the working electrode; the nanotubes on the other Mo electrodes 105 remain nitro-terminated (shown as Step C in FIG. 1).

Modification of Amino-Modified Nanotube Electrodes with DNA Oligonucleotides (Shown as Step D in FIG. 1):

The amino-terminated carbon nanotubes 106 were then made reactive toward 5′ thiol terminated oligonucleotides by immersing the chip in a 1.5 mM solution of the heterobifunctional cross-linker succinimidyl 4-(N-maleimidomethyl)cyclohexan-1-carboxylate (SSMCC, Pierce) in 0.1 M triethanolamine buffer (Aldrich), pH 7.0 for 20 minutes. The chip was then rinsed with DI water. The maleimide-terminated nanotubes were reacted with a 32 base oligonucleotide “S1” with the 5′ thiol modifier C6 (Glen Research) (5′-HS—C₆H₁₂T₁₅ AA CGA TCG AGC TGC AA-3′, UW biotechnology center.) The thiol modifier was deprotected and the oligonucleotide S1 was subsequently purified by reverse-phase HPLC and used immediately. S1 (50 μM in triethanolamine buffer, pH 7) was applied to the surface and kept in a humid chamber in the dark for two hours. The chip was rinsed in 2×SSPE/0.2% SDS buffer (Promega, consisting of 2 mM EDTA, 7 mM SDS, 300 mM NaCl, 20 mM NaH₂PO₄ with the pH 7.4) to remove excess DNA. At the end of this step, the nanotubes on a specific Mo electrode 101 have been covalently bonded to a specific biomolecule, such as DNA oligonucleotide S1.

To produce an array of distinct biologically-modified electrodes 107, the electrochemical reduction (previous step) followed by the covalent linking (the current step) can be repeated with a different biomolecule of interest. In this example the procedure was preformed a total of 4 times, producing a 4-element array of electrodes with distinct oligonucleotides (shown as Step E in FIG. 1). In each of the four cycles, the entire chip was exposed to all reactants and oligonucleotides—no mechanical barriers or specialized fluid handling was needed. Although only four distinguishable electrodes were produced in this example, in practice it is possible to repeat the process a very large number of times to produce a very high-density array of distinct biomolecular recognition sites.

Confirmation of Specific Biomolecular Recognition Properties of DNA-Modified Nanotube Electrodes:

In order to detect the surface-bound oligonucleotides and ascertain their biomolecular recognition capability, the chips were immersed in solutions containing fluorescently-labeled complementary DNA oligonucleotides. For example, to check the successful modification of the nanotubes with sequence S1 described above, the sample was exposed to the complementary sequence “F1” (5′-FAM-TT GCA GCT CGA TCG TT-3′) labeled with the fluorescent dye 6-FAM amidite (Glen Research) (5 μM in 2×SSPE/0.2% SDS buffer). The chip was subsequently rinsed in 2×SSPE/0.2% SDS buffer and the fluorescence intensity was imaged using either a fluorescence microscope or a fluorescence scanner (Genomic Solutions GeneTac UC4×4).

For the experiments involving four different DNA sequences covalently attached to four distinct regions (labeled 1-4 in FIG. 1) on the chip, the following sequences were used, using the same attachment chemistry as outlined above: 5′-HS—C₆H₁₂T₁₅ AA CGA TCG AGC TGC AA-3′ (S1); 5′-HS—C₆H₁₂T₁₅ AA CGA TCG AGG AGC AA-3′ (S2); 5′-HS—C₆H₁₂T₁₅ GC TTA TCG AGC TTT CG-3′ (S3); 5′-HS—C₆H₁₂T₁₅ GC TTA AGG AGC AAT CG-3′ (S4). To prepare the array of four different DNA sequences, the first region was electrochemically activated (i.e., the nitro groups were reduced to amino groups) and modified with S1 as described above. The next region on the chip was then electrochemically activated and then functionalized with S2, and similarly for subsequent regions, producing a 4-element array of DNA-modified nanotube electrodes in which each nanotube electrode was modified with a different sequence of DNA. To check the selectivity of local chemical modification, the entire chip that was modified with the four different sequences of DNA at four different locations was immersed in a mixture of all four of the complementary DNA sequences, 5′-FAM-TT GCA GCT CGA TCG TT-3′ (F1, complementary to S1); 5′-Cy3-TT GCT CCT GCA TCG TT-3′ (F2, complementary to S2); 5′-Cy3.5-CG AAA GCT CGA TAA GC-3′ (F3, complementary to S3); 5′-Cy5.5-CG ATT GCT CCT TAA GC-3′ (F4, complementary to S4), 5 μM of each in 2×SSPE/0.2% SDS buffer, for 20 minutes (shown as Step F in FIG. 1). As noted in the sequences of F1 through F4, each complementary sequence was modified with a different fluorescent tag. These were, specifically, 6-FAM amidite, Cy3 Phosphoramidite, Cy3.5 Phosphoramidite, and Cy5 Phosphoramidite. The fluorescent tags were purchased from Glen Research and the complete oligonucleotides were synthesized by the UW biotechnology center. The tags used have distinct enough peak absorbance and emission wavelengths such that each could be distinguished from the other on a single chip by fluorescence measurements.

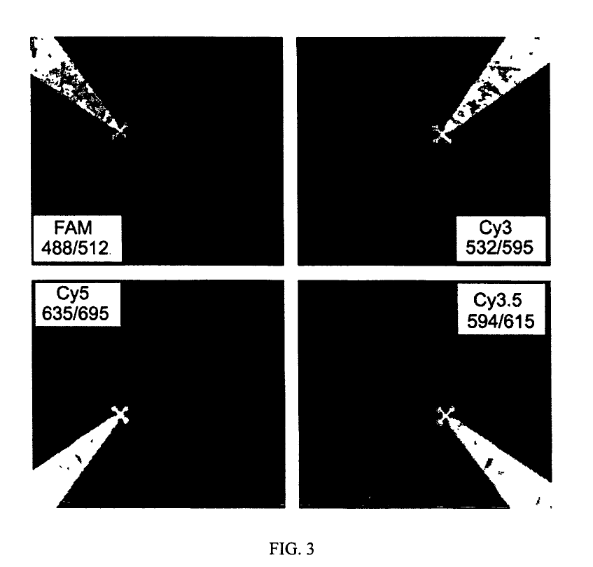

A fluorescence imager (Genomic Solutions GeneTac UC4×4) was used to generate a fluorescence image of the differentially modified regions on the chip by successive scanning with lasers and optical filters matching the appropriate absorption and emission profiles of the individual dyes. The specific excitation and emission wavelengths are as follows: 488 nm excitation and 512 nm emission bandpass filter for the FAM dye; 532 nm excitation and 595 nm bandpass filter for Cy3; 594 nm excitation and 615 nm bandpass filter for Cy3.5; and finally 635 nm excitation and 695 nm bandpass filter for Cy5. The resulting images are represented as grayscale intensity maps (one for each particular set of absorption/emission wavelengths optimized for a specific dye) in FIG. 3. However, the image could also be represented using colors approximately representing the true colors of the dyes.

The image of FIG. 3 shows that the complementary DNA molecules are able to recognize their appropriate complementary sequences with a high degree of selectivity, each sequence in solution hybridizing only with the complementary sequence bonded to the carbon nanotubes. Thus, the electrically-addressable modification process leads to biologically-modified electrodes exhibiting a high degree of biological specificity.

### Example 2

### Production of a Microsensor from Diamond Electrodes

This example describes the principal for production of a biosensor array from individually electrically addressable diamond electrodes. A polycrystalline diamond sample was cleaned by immersion in a hydrogen plasma to produce a diamond surface terminated with hydrogen atoms. This hydrogen-terminated diamond sample was then immersed in a 36 mM solution of 4-nitrobenzenediazonium tetrafluoroborate (Aldrich,) in 1% sodium dodecyl sulfate (Promega, Madison, Wis.) and shaken using a vortex mixer at room temperature for 24 hours to provide a nitro-modified diamond surface.

The nitro groups on the nitro-modified surface were electrochemically reduced to produce amino groups, using the method described above for the Mo/carbon nanotube electrodes of Example 1. The resulting amino-modified diamond electrode was then reacted with the DNA oligonucleotide S4 (5′-HS—C₆H₁₂T₁₅ GC TTA AGG AGC AAT CG-3′) using the same procedure described in Example 1, above.

In order to detect the surface-bound oligonucleotides and ascertain their biomolecular recognition capabilities, fluorescence experiments were conducted by immersing the electrode in solutions containing fluorescently-labelled complementary DNA oligonucleotides, as described in Example 1. When exposed to a fluorescently-labeled oligonucleotide with the complementary sequence (i.e., sequence F4 described above), a high intensity of fluorescence was observed from the sample. A control sample that was went through an identical procedure except that the electrochemical reduction step was not performed, yielded almost no fluorescence.

### Example 3

### Production of a Microsensor Array from Vertically Aligned Carbon Nanotubes

To test the ability to achieve high spatial resolution with the present methods, experiments were conducted on electrodes composed of ˜500 nm-diameter bundles of vertically aligned carbon nanofibers (VACNs) on Mo contacts.

Substrate Patterning:

In these experiments, Mo electrodes were fabricated on silicon wafers (Si<100>) covered with a 300 nm thick film of low-pressure chemical vapor deposited silicon nitride as an insulator. Molybdenum contacts were then patterned onto the substrate as described in Example 1. Electron beam lithography was used with PMMA photoresist to define small regions for the selective deposition of catalyst onto the patterned regions of the substrate; the catalyst consisted of a thin film of 20 nm titanium and 20 nm nickel.

Nanofiber Growth:

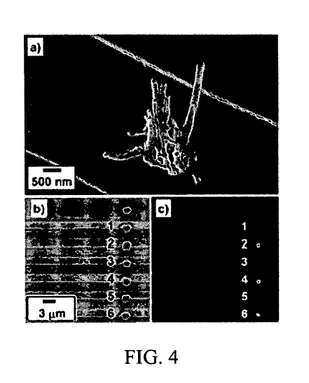

VACNs with typical diameters of of 50-100 nm each were grown on the catalyst-patterned substrate using 4 torr of a mixture of acetylene (16 sccm flow rate) and ammonia (80 sccm) using a home-built DC Plasma-Enhanced Chemical Vapor Deposition reactor. With the sample as the cathode, 330 watts of power was applied to the sample for 12 minutes using an Advanced Energy MDX 1K power supply; the plasma heated the sample to approximately 700° C. More detail regarding the production of VACNs may be found in Cruden et al., J. Appl. Phys, 94, 4070-4078 (2003), the entire disclosure of which is incorporated herein by reference. FIG. 4a shows a scanning electron microscope image of a ˜500 nm diameter bundle consisting of approximately ˜10 nanofibers, grown on a 3 micron wide Mo electrode. FIG. 4b shows a white-light image of this same sample, showing the six Mo electrodes on which carbon nanofiber bundles were grown. The nanofiber bundles appear as dark, resolution-limited fuzzy patches in the optical microscopy image.

Nanofiber Functionalization and Modification:

The VACNs were functionalized with aromatic nitro groups, electrochemically modified to provide amino-functionalized VACNs and reacted with DNA oligonucleotides using the same procedures described in Example 1 for the single-walled carbon nanotubes. In this example, only the nanofibers on electrodes 2, 4, and 6 were functionalized using the electrochemical modification process, and the remaining three electrodes were left alone as a control. FIG. 4c shows a fluorescence image after selective electrochemical modification, functionalization with DNA sequence S1, described in Example 1, and exposure to the fluorescein-labeled complement F1, described in Example 1. As expected, the fluorescence image shows only those nanofiber bundles that were selectively reduced; virtually no fluorescence is observed for the other bundles on the Mo electrodes or from one bundle grown on the silicon nitride insulator. These results demonstrate that it is possible to use the method described here to electrochemically address and functionalize VACNs with biomolecules such as DNA on sub-micron length scales.

It is understood that the invention is not confined to the particular embodiments set forth herein, but embraces all such forms thereof as come within the scope of the following claims.

What is claimed is:

1. A method for selectively modifying electrodes derivatized with a first functional group in an array of electrically addressable electrodes, the method comprising:
(a) applying a potential to at least one electrically addressable electrode to electrochemically reduce the first functional group to provide a second functional group; and
(b) exposing the second functional group to a binding entity that reacts with the second functional group but not with the first functional group.

2. The method of claim 1 wherein the first functional group is a nitro group and the second functional group is an amino group.

3. The method of claim 1 wherein the electrically addressable electrodes comprise a carbon-containing material.

4. The method of claim 1 wherein the array of electrically addressable electrodes comprises an array of electrically conductive contacts having carbon nanotubes disposed thereon.

5. The method of claim 4 wherein the first functional group is a nitro group and the second functional group is an amino group.

6. The method of claim 4 wherein the contacts comprise molybdenum contacts.

7. The method of claim 1 wherein the array of electrically addressable electrodes comprises an array of electrically conductive contacts having vertically aligned carbon nanofibers disposed thereon.

8. The method of claim 3 wherein the carbon-containing material comprises diamond.

9. The method of claim 3 wherein the carbon-containing material comprises glassy carbon.

10. The method of claim 3 wherein the carbon-containing material comprises diamond-like carbon.

11. The method of claim 3 wherein the carbon-containing material comprises graphitic carbon.

12. The method of claim 3 wherein the carbon-containing material comprises a conductive polymer.

13. The method of claim 1 wherein the binding entities comprising sensor molecules having specific affinities for analyte molecules.

14. The method of claim 13 wherein the sensor molecules comprise biomolecules.

15. The method of claim 14 wherein the biomolecules comprise oligonucleotides.

16. The method of claim 13 wherein the sensor molecules are selected from the group consisting of DNA molecules, RNA molecules, synthetic oligonucleotides, peptides, polypeptides, proteins, enzymes, antibodies, receptors, polysaccharides, synthetic polymers, ligands and viruses.

17. The method of claim 13 wherein the binding entities comprise a spacer molecule bound to the sensor molecule.

18. The method of claim 17 wherein the first functional group is a nitro group, the second functional group is an amino group, and the binding entity comprises the reaction product of a succinimidyl 4-(N-maleimidomethyl)cyclohexan-1-carboxylate and an oligonucleotide modified with a thiol group at its 5′ end.

19. The method of claim 13, wherein the second functional groups react with the spacer molecules and the spacer molecules subsequently react with the sensor molecules.

20. A sensor array comprising:
(a) an array of electrically addressable electrodes disposed on a substrate, the electrically addressable electrodes comprising electrically conductive contacts having one or more carbon nanotubes disposed thereon; and
(b) one or more binding entities bound to the electrically addressable electrodes, the binding entities comprising sensor molecules having specific affinities for analyte molecules.

21. The sensor array of claim 20, comprising at least 10 electrically addressable electrodes.

22. The sensor array of claim 20, comprising at least 1000 electrically addressable electrodes.

23. The sensor array of claim 20 wherein the one or more carbon nanotubes comprise a bundle of vertically aligned carbon nanofibers.

24. The sensor array of claim 20 wherein the electrically addressable electrodes comprise individually electrically addressable electrodes.

25. The sensor array of claim 20 wherein the sensor molecules comprise biomolecules.

26. The sensor array of claim 20 wherein the sensor molecules are selected from the group consisting of DNA molecules, RNA molecules, synthetic oligonucleotides, peptides, polypeptides, proteins, enzymes, antibodies, receptors, polysaccharides, synthetic polymers, ligands and viruses.

27. The sensor array of claim 20 wherein the binding entities comprise spacer molecules bound to sensor molecules.

28. The sensor array of claim 27 wherein the binding entities comprise the reaction product of a succinimidyl 4-(N-maleimidomethyl)cyclohexan-1-carboxylate molecule and an oligonucleotide modified with a thiol group at its 5′ end.

29. An sensor array comprising:
(a) an array of electrically addressable electrodes disposed on a substrate, the electrically addressable electrodes comprising an electrically conductive material consisting essential of elemental carbon; and
(b) one or more binding entities bound to the electrically addressable electrodes, the binding entities comprising sensor molecules having specific affinities for analyte molecules.

30. The sensor array of claim 29, comprising at least 10 electrically addressable electrodes.

31. The sensor array of claim 29, comprising at least 1000 electrically addressable electrodes.

32. The sensor array of claim 29 wherein the carbon-containing material is diamond.

33. The sensor array of claim 29 wherein the carbon-containing material is glassy carbon.

34. The sensor array of claim 29 wherein the carbon-containing material is graphitic carbon.

35. The sensor array of claim 29 wherein the sensor molecules comprise biomolecules.

36. The sensor array of claim 29 wherein the sensor molecules are selected from the group consisting of DNA molecules, RNA molecules, synthetic oligonucleotides, peptides, polypeptides, proteins, enzymes, antibodies, receptors, polysaccharides, synthetic polymers, ligands and viruses.

37. The sensor array of claim 29 wherein the binding entities comprise a spacer molecule bound to the sensor molecule.

38. An sensor array comprising:
(a) an array of electrically addressable electrodes disposed on a substrate, the electrically addressable electrodes comprising an electrically conductive carbon-containing material comprising diamond or graphitic carbon; and
(b) one or more binding entities bound to the electrically addressable electrodes, the binding entities comprising sensor molecules having specific affinities for analyte molecules.

39. The sensor array of claim 38 wherein the carbon-containing material comprises diamond.

40. The sensor array of claim 38 wherein the sensor molecules comprise biomolecules.

41. The sensor array of claim 38 wherein the sensor molecules are selected from the group consisting of DNA molecules, RNA molecules, synthetic oligonucleotides, peptides, polypeptides, proteins, enzymes, antibodies, receptors, polysaccharides, synthetic polymers, ligands and viruses.

42. The sensor array of claim 38 wherein the binding entities comprise a spacer molecule bound to the sensor molecule.

43. A modified surface comprising a substrate surface, the substrate surface comprising at least two surface regions each functionalized with a binding-entity, wherein the functionalized surface regions have surface areas of less than 1 micron and further wherein the binding entities of the at least two regions are separated by less than about 10 microns.

44. The modified surface of claim 43 wherein the binding entities of the at least two regions are separated by no more than about 1 micron.

45. The modified surface of claim 43 wherein the binding entity-functionalized surface regions have a surface area of no more than 0.5 microns.

46. The modified surface of claim 43 wherein the functionalized surface regions comprise binding entity-functionalized vertically aligned carbon nanofibers disposed on the substrate surface.

47. The modified surface of claim 43 wherein the binding entities comprise sensor molecules having specific affinities for analyte molecules.

48. The modified surface of claim 47 wherein the sensor molecules comprise biomolecules.

49. The modified surface of claim 47 wherein the sensor molecules are selected from the group consisting of DNA molecules, RNA molecules, synthetic oligonucleotides, peptides, polypeptides, proteins, enzymes, antibodies, receptors, polysaccharides, synthetic polymers, ligands and viruses.
